(19) United States
(12) Patent Application Publication
(10) Pub. No.: US 2025/0367245 A1
(43) Pub. Date: Dec. 4, 2025

(54) METHOD FOR DELAYING MUSCLE LOSS, IMPROVING MUSCLE ENDURANCE, OR DELAYING CELL AGING BY USING LIMOSILACTOBACILLUS REUTRI AND/OR METABOLITES THEREOF

(71) Applicant: TCI CO., LTD., Taipei (TW)

(72) Inventors: YUNG-HSIANG LIN, Taipei (TW); JIN-JIA WANG, Taipei (TW); YI-RONG LI, Taipei (TW)

(21) Appl. No.: 19/177,652

(22) Filed: Apr. 14, 2025

Related U.S. Application Data
(60) Provisional application No. 63/636,114, filed on Apr. 19, 2024.

(51) Int. Cl. A61K 35/744; A61P 21/00

(52) U.S. Cl. CPC A61K 35/744; A61P 21/00

(57) ABSTRACT

Method for delaying muscle loss, improving muscle endurance or delaying cell aging, comprising: administering to a subject in need an effective dose of the Limosilactobacillus reuteri and/or It's Metabolite. The Limosilactobacillus reuteri was deposited at Food Industry Research and Development Institute under the accession number BCRC911170 or erman Collection of Microorganisms and Cell Cultures under the accession number DSM34539.

### CROSS-REFERENCE TO RELATED APPLICATION

This application claims the benefit of U.S. provisional application Ser. No. 63/636,114, filed on Apr. 19, 2024. The entirety of the above-mentioned patent application is hereby incorporated by reference herein and made a part of the specification.

### REFERENCE OF AN ELECTRONIC SEQENCE LISTING

The contents of the electronic sequence listing (P245887USI.xml; Size: 3,211 bytes; and Date of Creation: Apr. 10, 2025) is herein incorporated by reference in its entirety.

### BACKGROUND

### Technical Field

The present invention relates to a Limosilactobacillus reuteri, and in particular, to use of a Limosilactobacillus reuteri and/or a metabolite thereof for preparation of a composition for delaying muscle loss, improving muscle endurance, or delaying cell aging.

### Related Art

Limosilactobacillus reuteri, an important bacterial species among lactic acid bacteria, is generally used to improve the intestinal microbiota of mammals and antagonize the colonization of other harmful bacteria in the intestine and serves as a probiotic with development potential.

### SUMMARY

The present invention provides a Limosilactobacillus reuteri and/or a metabolite thereof, where the Limosilactobacillus reuteri is Limosilactobacillus reuteri TCI979 with the accession number BCRC911170 or DSM34539.

In some embodiments, a method for delaying muscle loss includes administering to a subject in need thereof a composition containing an effective dose of a Limosilactobacillus reuteri and/or a metabolite thereof. The Limosilactobacillus reuteri and/or the metabolite thereof are used for preparation of a composition for delaying the muscle loss.

In some embodiments, a method for improving muscle endurance includes administering to a subject in need thereof a composition containing an effective dose of a Limosilactobacillus reuteri and/or a metabolite thereof. The Limosilactobacillus reuteri and/or the metabolite thereof are used for preparation of a composition for improving the muscle endurance.

In some embodiments, a method for delaying cell aging includes administering to a subject in need thereof a composition containing an effective dose of a Limosilactobacillus reuteri and/or a metabolite thereof. The Limosilactobacillus reuteri and/or the metabolite thereof are used for preparation of a composition for delaying the cell aging.

In some embodiments, the Limosilactobacillus reuteri may reduce a content of malondialdehyde in blood.

In some embodiments, the Limosilactobacillus reuteri may improve total antioxidant capacity (TAC) or a content of sulfur-containing compounds (f-Thiols) in blood.

In some embodiments, the Limosilactobacillus reuteri may inhibit a content of reactive oxygen.

In some embodiments, the Limosilactobacillus reuteri may reduce apoptosis.

In some embodiments, the Limosilactobacillus reuteri may improve mitochondrial activity of muscle cells.

In some embodiments, the Limosilactobacillus reuteri may delay a rate of telomere shortening.

In some embodiments, the Limosilactobacillus reuteri may improve activity of telomerase.

In summary, the Limosilactobacillus reuteri and/or the metabolite thereof in any of the embodiments may reduce the content of malondialdehyde in blood, improve the total antioxidant capacity or the content of sulfur-containing compounds in blood, inhibit the content of reactive oxygen, or reduce the apoptosis, thereby delaying the muscle loss. The Limosilactobacillus reuteri and/or the metabolite thereof in any of the embodiments may improve the mitochondrial activity of muscle cells, reduce the content of malondialdehyde in blood, improve the total antioxidant capacity or the content of sulfur-containing compounds in blood, or inhibit the content of reactive oxygen, thereby improving the muscle endurance. The Limosilactobacillus reuteri and/or the metabolite thereof in any of the embodiments may delay the rate of telomere shortening, improve the activity of telomerase, or inhibit the content of reactive oxygen, thereby delaying the cell aging.

### BRIEF DESCRIPTION OF THE DRAWINGS

FIG. 1 is a result graph of a test for Limosilactobacillus reuteri in inhibiting ROS.

FIG. 2 is a result graph of a test for Limosilactobacillus reuteri in preventing apoptosis.

FIG. 3 is a result graph of a test for Limosilactobacillus reuteri in inhibiting low mitochondrial activity.

FIG. 4 is a result graph of a test for Limosilactobacillus reuteri in improving telomerase activity.

FIG. 5 is a result graph of a test for Limosilactobacillus reuteri in changing cell size.

FIG. 6 shows microscopic observation result images of a change in cell size by Limosilactobacillus reuteri under natural aging.

FIG. 7 is a result graph of a test for Limosilactobacillus reuteri in changing cell size under external oxidative damage.

FIG. 8 is a result graph of changes in a concentration of MDA in blood in a human experiment.

FIG. 9 is a result graph of changes in total antioxidant capacity in a human experiment.

FIG. 10 is a result graph of changes in a concentration of sulfur-containing compounds in blood in a human experiment.

FIG. 11 is a result graph of changes in average upper limb muscle endurance in a human experiment.

FIG. 12 is a result graph of changes in average lower limb muscle endurance in a human experiment.

### DETAILED DESCRIPTION

Limosilactobacillus reuteri TCI979 has been deposited at the Food Industry Research and Development Institute, a foundation corporation, under the accession number BCRC911170, and at the German Collection of Microorganisms and Cell Cultures under the accession number DSM34539. In some embodiments, Limosilactobacillus reuteri TCI979 is a strain isolated from Wolongtan in Guizhou, China.

The Limosilactobacillus reuteri of the present invention, also known as Limosilactobacillus reuteri TCI979, is commonly found in the intestines, oral cavities, and stomachs of humans and animals. It may also be present in fermented foods (such as yogurt and pickles) and dairy products. Here, Limosilactobacillus reuteri TC1979 is a facultative anaerobic Gram-positive bacterium, but it has a greater preference for a hypoxic or anaerobic environment. It is generally elongated and curved rod-shaped in appearance, with blunt ends. Its size is approximately 0.5 to 0.8 micrometers in width and 2 to 8 micrometers in length. It lacks flagella and thus has no active motility ability. It does not produce spores and generally does not form a capsule. Its main energy source is carbohydrates, such as monosaccharides (such as glucose and fructose) and disaccharides (such as lactose), which serve as a carbon source. It metabolizes glucose through the heterolactic fermentation pathway (phosphoketolase pathway), producing metabolites such as lactic acid, ethanol, acetic acid, and carbon dioxide. Limosilactobacillus reuteri may metabolize glycerol to produce reuterin. Reuterin is a non-protein antibacterial substance that may effectively inhibit the growth of harmful bacteria and is harmless to animals.

In some embodiments, Limosilactobacillus reuteri TC1979 and/or a metabolite thereof have the effect of delaying muscle loss. In other words, the Limosilactobacillus reuteri TC1979 and/or the metabolite thereof are suitable for preparation of a composition for delaying the muscle loss.

In some embodiments, Limosilactobacillus reuteri TC1979 and/or a metabolite thereof have the effect of improving muscle endurance. In other words, the Limosilactobacillus reuteri and/or the metabolite thereof are used for preparation of a composition for improving the muscle endurance.

In some embodiments, Limosilactobacillus reuteri TC1979 and/or a metabolite thereof have the effect of delaying cell aging. In other words, the Limosilactobacillus reuteri and/or the metabolite thereof are used for preparation of a composition for delaying the cell aging.

In some embodiments, the Limosilactobacillus reuteri may reduce a content of malondialdehyde in blood. Malondialdehyde, abbreviated as MDA, is generally formed due to lipid peroxidation. Malondialdehyde is highly active in organisms and easily binds to DNA or proteins, thus disrupting the development of normal cells. Generally, the higher the content of malondialdehyde in the body, the poorer the antioxidant capacity. At the same time, studies have shown that with the increase of age, the content of MDA in muscles rises significantly, and it gradually increases as the muscle mass decreases.

In some embodiments, Limosilactobacillus reuteri may improve total antioxidant capacity (abbreviated as TAC). The total antioxidant capacity refers to the ability of the body to reduce oxides. The higher the TAC, the more it can mitigate the damage caused by reactive oxygen free radicals, representing the antioxidant capacity of the body. Improving the antioxidant capacity may further improve muscle strength and muscle mass.

In some embodiments, Limosilactobacillus reuteri may increase a content of sulfur-containing compounds (f-Thiols) in blood. Here, the sulfur-containing compounds refer to compounds containing sulfur in blood. The sulfur-containing compounds can act as free radical scavengers, directly neutralize free radicals, or reduce oxidative stress by regulating the redox signaling pathway (such as increasing the activity of glutathione reductase). Reducing oxidative stress helps maintain the normal functions of cells, including energy metabolism, DNA repair, and protein synthesis, and improves muscle strength and mass.

In some embodiments, the Limosilactobacillus reuteri may inhibit a content of reactive oxygen.

In some embodiments, the Limosilactobacillus reuteri may reduce apoptosis. Apoptosis is a natural cell death phenomenon that occurs under the regulation of genes after cells are stimulated by the environment, so it is also known as programmed cell death. It is different from cell necrosis. Under normal circumstances, any abnormalities that occur during the formation of cells will be eliminated through apoptosis. For example, the process of cancer cells in the body growing into tumors will be inhibited under the guidance of apoptosis.

In some embodiments, the Limosilactobacillus reuteri may improve mitochondrial activity of muscle cells.

In some embodiments, the Limosilactobacillus reuteri may delay a rate of telomere shortening. In some embodiments, the Limosilactobacillus reuteri may improve activity of telomerase. Telomeres refer to highly repetitive DNA sequences or protein complexes located at the ends of chromosomes, which are crucial for maintaining the integrity and stability of chromosomes. It means that when the length of telomeres shortens with cell division, this shortening phenomenon is prone to causing cell aging or diseases, and telomerase can more effectively lengthen and maintain the length of telomeres, delaying the rate of telomere shortening.

In some embodiments, a specific content of the Limosilactobacillus reuteri is 100 mg/day.

In some embodiments, the aforementioned composition includes a specific content of the Limosilactobacillus reuteri or the metabolite thereof. It means that a dosage of the Limosilactobacillus reuteri TC1979 is 100 mg/day. Specifically, assuming that one portion of the composition is administered daily and the Limosilactobacillus reuteri is in the form of a dry powder, the composition contains at least 100 mg of the Limosilactobacillus reuteri.

In some embodiments, the aforementioned composition may be a health product, food product, or food additive for a non-medical purpose. In other words, this health product, food product, or food additive includes a specific dosage of Limosilactobacillus reuteri.

In some embodiments, the aforementioned health product, food product, or food additive may further contain a food industry acceptable carrier that is widely used in food manufacturing technology. For example, the food industry acceptable carrier may contain one or more of the following reagents: a solvent, a buffer, an emulsifier, a suspending agent, a decomposer, a disintegrating agent, a dispersing agent, a binding agent, an excipient, a stabilizing agent, a chelating agent, a diluent, a gelling agent, a preservative, a wetting agent, a lubricant, an absorption delaying agent, a liposome, and the like. The selection and quantity of these reagents fall within the scope of the professional competence and routine techniques of those skilled in the art.

In some embodiments, the acceptable carrier of the aforementioned health product, food product, or food additive includes a solvent selected from the group consisting of water, normal saline, phosphate buffered saline (PBS), and an aqueous solution containing alcohol.

In some embodiments, the food product can be but is not limited to: a beverage, a fermented food, a bakery product, a health food for a non-medical purpose, and a dietary supplement.

### Example 1: Isolation and Identification of Bacterial Species

First, an appropriate amount of the water from Wolongtan in Guizhou, China, was added to the liquid Lactobacilli MRS medium (BD Difco™ Lactobacilli MRS Broth) and cultured for 24 hours at 37° C. in an anaerobic environment (i.e., the oxygen concentration in the culture environment was 1% by volume) to form a bacterial solution. Next, after performing serial dilution on the bacterial solution, it was spread-plated onto the solid Lactobacilli MRS medium (BD Difco™ Lactobacilli MRS Broth with 1.5% agar) and cultured at 37° C. in an anaerobic environment (i.e., the oxygen concentration in the culture environment was 1% by volume) until colonies formed on the culture plate. One of the colonies was selected for subsequent testing.

Then, the strain of the colony was identified for its bacterial species. The 16S ribosomal gene (16S rDNA) sequence (i.e., SEQ ID NO: 1) of this isolated strain was obtained through polymerase chain reaction (PCR), and the total length of the gene sequence was 1,050. Next, using the website of the National Center for Biotechnology Information (NCBI), after comparing the shown gene sequence with the 16S rDNA sequences of other Lactobacillus species, the similarities of the 16S rDNA sequences are shown in Table 1 below. Based on this, it was found that the 16S rDNA sequence of this isolated strain had the highest similarity to that of Limosilactobacillus reuteri (accession number NBRC 15892).

TABLE 1

| | | Similarity |
|---|---|---|
| Comparison strain number | Bacterial species | (%) |
| Limosilactobacillus reuteri | Limosilactobacillus reuteri | 99.42% |
| strain NBRC 15892 | | |
| Limosilactobacillus portuensis | Limosilactobacillus portuensis | 98.92% |
| strain c11Ua_112_M | | |
| Limosilactobacillus equigenerosi | Limosilactobacillus equigenerosi | 95.67% |
| strain NRIC 0697 | | |
| Limosilactobacillus fermentum | Limosilactobacillus fermentum | 95.00% |
| strain CIP 102980 | | |
| Limosilactobacillus vaginalis | Limosilactobacillus vaginalis | 93.60% |
| strain NCTC 12197 | | |

Here, the 16S rDNA sequence of the strain isolated from the water of Wolongtan in Guizhou, China, had a high similarity of 99.42% to the sequence of Limosilactobacillus reuteri. Therefore, this isolated strain was named Limosilactobacillus reuteri TCI979.

### Example 2: Preservation and Cultivation Experiment of Limosilactobacillus reuteri

The isolated Limosilactobacillus reuteri was inoculated into MRS medium (BD Difco™ Lactobacilli MRS Broth, 1% (v/v)) at an inoculum amount of 1% (approximately 1×10⁴ CFU/mL), and cultured for 24 hours at 37° C. in an anaerobic environment to form a TC1979 bacterial solution.

The TCI979 bacterial solution was centrifuged at a speed of 5000 rpm for 20 minutes to separate the supernatant and the cells of Limosilactobacillus reuteri. The supernatant was filtered through a 0.2 μm filter membrane, and the obtained filtrate was the TCI979 sample (i.e., the TC1979 sample contains the metabolite of the Limosilactobacillus reuteri).

The above-mentioned TCI979 bacterial solution was added with 5% (W/W) soybean milk powder, 5% (W/W) trehalose, 10% (W/W) sorbitol, and 20% (W/W) indigestible maltodextrin, mixed evenly, freeze-dried, and ground into powder to form the TCI979 bacterial powder.

### Example 3: Test of Reactive Oxygen Content

In this test, hydrogen peroxide (H₂O₂) was used to simulate the situation where cells are subjected to external oxidative damage, and whether the sample could assist the cells in resisting oxidative damage was observed. Here, the probe DCFH-DA was used in combination with a flow cytometer to determine the change in the content of reactive oxygen species (ROS) in human peripheral blood mononuclear cells after being treated with the TCI979 sample.

### 3-1. Materials and Instruments:

Cells: human peripheral blood mononuclear cells (purchased from Lifeline, model HC-002, hereinafter referred to as PBMC cells for short).

Medium: RPMI 1640 (Roswell Park Memorial Institute, purchased from Gibco), added with 10% FBS (Fetal Bovine Serum, purchased from Gibco) and 1% antibiotic-antifungal agent (purchased from Gibco).

PBS buffer and DPBS buffer (purchased from Gibco).

Hydrogen peroxide H₂O₂ (purchased from Sigma).

2,7-dichloro-dihydro-fluorescein diacetate (DCFH-DA) (purchased from Sigma/SI-D6883-50 MG). Before use, the DCFH-DA was dissolved in DMSO to be diluted into a DCFH-DA solution with a concentration of 5 μg/mL.

Flow cytometer (purchased from BD Company, model Accuri™ C6 Plus).

### 3-2. Test Procedure:

First, 1×10⁵ PBMC cells were taken into a six-well cell culture plate containing 2 mL of medium per well and cultured at a constant temperature of 37° C. and 5% CO₂ for 24 hours.

When the PBMC cells in each well were in a suspended state in the cell culture dish, the PBMC cells were separated into a blank group, a control group, and an experimental group.

Blank group: only the medium was added and treated at 37° C. for 2 hours.

Control group: the medium was added and treated at 37° C. for 1 hour, then 1 mM of hydrogen peroxide was added, and the treatment was continued at 37° C. for 1 hour.

Experimental group: the medium containing the TCI979 sample prepared in Example 2 with a concentration of 0.125% was added and treated at 37° C. for 1 hour, then 1 mM of hydrogen peroxide was added, and the treatment was continued at 37° C. for 1 hour.

Next, 5 μg/mL DCFH-DA solution was added to the cells of each group and treated at 37° C. for 15 minutes.

The cells of each group were transferred to a 1.5 mL centrifuge tube, centrifuged (400×g) for 5 minutes, the supernatant was removed, washed with 1× PBS buffer, and then centrifuged (400×g) for 5 minutes again. After centrifugation, the supernatant was removed, 200 μL of 1× DPBS buffer was added to each centrifuge tube to resuspend the PBMC cells, and then a cell solution to be tested was formed.

The flow cytometer (set excitation light: 450-490 nm; scattered light: 510-550 nm) was used to detect the fluorescence signal of DCFH-DA in the cell solution to be tested of each group. The fluorescence intensity of the PBMC cells treated with DCFH-DA can reflect the content of reactive oxygen species (ROS) in the cells and was converted into a relative value.

### 3-3. Test Results:

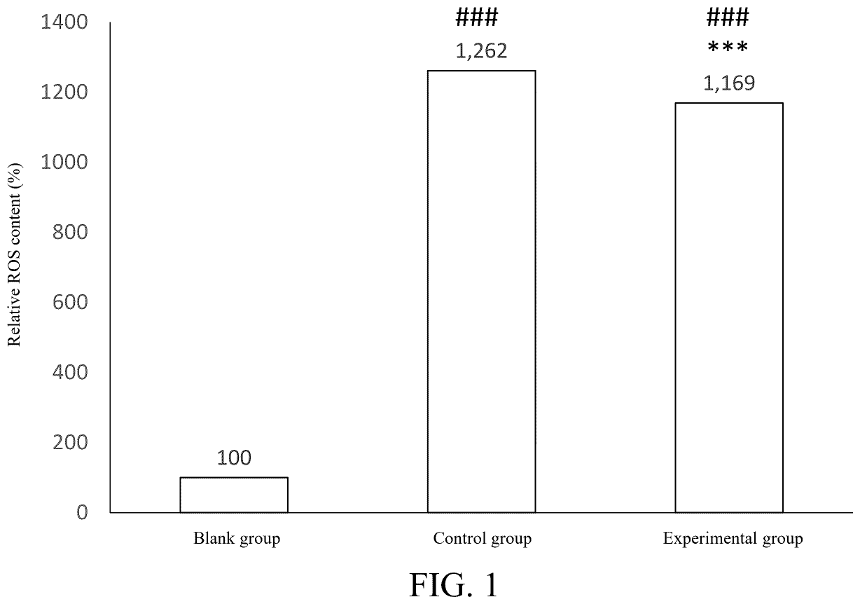

It should be particularly noted that the results shown in FIG. 1 were presented in relative multiples, i.e., the quantitative result of the blank group was regarded as 100, and the quantitative results of the experimental group and the control group were converted into the expression amount relative to that of the blank group. The Student T-Test was used to analyze whether there was a statistically significant difference. In the figure, “#” represents that the p-value is less than 0.05 relative to the blank group, “##” represents that the p-value is less than 0.01 relative to the blank group, and “###” represents that the p-value is less than 0.001 relative to the blank group; “*” represents that the p-value is less than 0.05 relative to the control group, “*” represents that the p-value is less than 0.01 relative to the control group, and “***” represents that the p-value is less than 0.001 relative to the control group.

Please refer to FIG. 1. As shown in FIG. 1, the control group showed that the simulation of oxidative damage could indeed induce the production of a large amount of ROS in the cells, and the ROS content in the control group increased by 1262% compared with that in the blank group. In contrast, the ROS content in the experimental group was 1169%, which decreased by 93% compared with that in the control group. It can be seen that Limosilactobacillus reuteri TC1979 can effectively assist cells in resisting the oxidative damage caused by hydrogen peroxide. It is known that a high concentration of ROS triggers oxidative stress, which leads to mitochondrial dysfunction, and then damages the components of skeletal muscle cells. At the same time, an increase in ROS interferes with the differentiation of skeletal muscle and reduces the generation efficiency of myoblasts.

### Example 4: Apoptosis Test

In this test, hydrogen peroxide (H₂O₂) was used to simulate the situation where cells are subjected to external oxidative damage, and whether the sample could assist the cells in avoiding apoptosis was observed. Here, Annexin V labeled with FITC fluorescence was used to stain the cells, and the nuclear stain PI was used in combination. At the same time, a fluorescence microscope or a flow cytometer was adopted to detect the phenomenon of cell apoptosis and the stages of apoptosis.

### 4-1. Materials and Instruments:

Cells: human mononuclear cells (obtained from ATCC, TIB202, hereinafter referred to as THP-1 cells for short).

Medium: RPMI 1640 (Roswell Park Memorial Institute, purchased from Gibco), added with 10% FBS (Fetal Bovine Serum, purchased from Gibco) and 1% antibiotic-antifungal agent (purchased from Gibco).

PBS buffer and DPBS buffer (purchased from Gibco).

Hydrogen peroxide H₂O₂ (purchased from Sigma).

Nuclear stain (propidium iodide, abbreviated as PI stain, purchased from BD, Bioscience).

Apoptosis test kit (Annexin V, FITC Apoptosis Detection Kit, purchased from Enzo).

Flow cytometer (purchased from BD Company, model Accuri™ C6 Plus). 4-2. Test Procedure:

First, 1×10⁵ THP-1 cells were seeded into a 24-well cell culture plate and cultured at a constant temperature of 37° C. and 5% CO₂ for 24 hours. The THP-1 cells were separated into a blank group, a control group, and an experimental group.

Blank group: only the medium was added and treated at 37° C. for 2 hours.

Control group: only the medium was added and treated at 37° C. for 1 hour, then 1 mM of hydrogen peroxide was added, and the treatment was continued at 37° C. for 1 hour.

Experimental group: the medium containing the TCI979 sample prepared in Example 2 with a concentration of 0.125% was added and treated at 37° C. for 1 hour, then 1 mM of hydrogen peroxide was added, and the treatment was continued at 37° C. for 1 hour.

The supernatant in the culture plate was removed and rinsed twice with 1 mL of 1× PBS solution. Next, the PI stain and the reagent (Annexin V) in the test kit were added to the cells of each group and treated at 37° C. for 5 minutes.

The cells of each group were transferred to a 1.5 mL centrifuge tube, centrifuged (400×g) for 5 minutes, the supernatant was removed, washed with 1× PBS buffer, and then centrifuged (400×g) for 5 minutes again. After centrifugation, the supernatant was removed, 200 μL of 1× DPBS buffer was added to each centrifuge tube to resuspend the PBMC cells, and then a cell solution to be tested was formed.

The flow cytometer (set excitation light: 450-490 nm; scattered light: 510-550 nm) was used to detect the fluorescence signals of FITC and PE in the cell solution to be tested of each group. The fluorescence intensity of the cells can reflect the situation of cell apoptosis and was converted into a relative value.

### 4-3. Test Results:

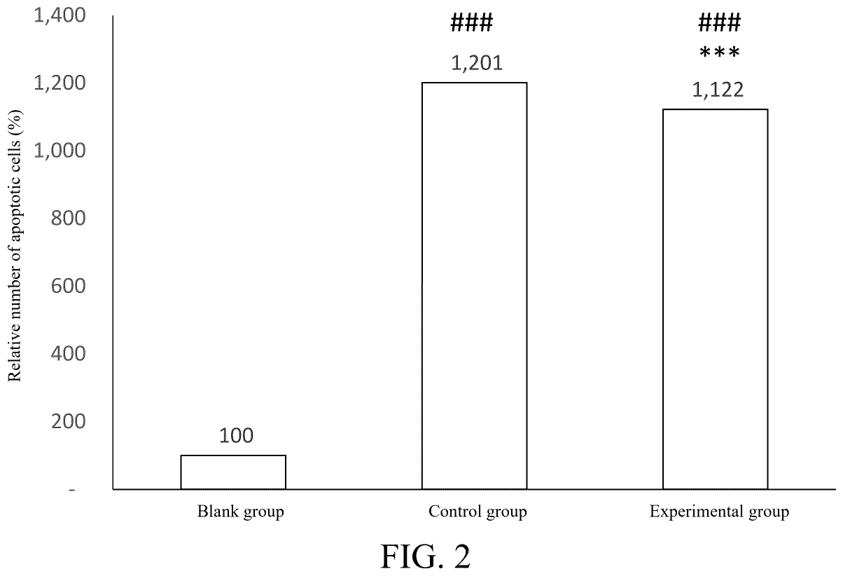

It should be particularly noted that the results shown in FIG. 2 were presented in relative multiples, i.e., the quantitative result of the blank group was regarded as 100, and the quantitative results of the experimental group and the control group were converted into the expression amount relative to that of the blank group. The Student T-Test was used to analyze whether there was a statistically significant difference. In the figure, “#” represents that the p-value is less than 0.05 relative to the blank group, “##” represents that the p-value is less than 0.01 relative to the blank group, and “###” represents that the p-value is less than 0.001 relative to the blank group; “*” represents that the p-value is less than 0.05 relative to the control group, “**” represents that the p-value is less than 0.01 relative to the control group, and “***” represents that the p-value is less than 0.001 relative to the control group.

Please refer to FIG. 2. As shown in FIG. 2, the control group showed that the simulation of oxidative damage could indeed cause a large number of cell apoptosis, and the number of apoptotic cells in the control group increased by 1201% compared with that in the blank group. In contrast, the number of apoptotic cells in the experimental group was 1122%, which decreased by 79% compared with that in the control group. It can be seen that Limosilactobacillus reuteri TC1979 can effectively assist cells in avoiding apoptosis under external damage. That is, Limosilactobacillus reuteri TC1979 can alleviate the apoptosis caused by oxidative stress, with an inhibition rate of 79%.

### Example 5: Experiment on Mitochondrial Activity in Skeletal Muscle Cells

Mitochondria are important organelles for cellular oxidative metabolism and energy supply. Poor mitochondrial function will lead to insufficient ATP production. Inactive and inefficient mitochondria are a kind of cellular waste. Excessive accumulation of mitochondria will lead to the aging phenomenon of cell swelling, thus reducing muscle exercise endurance. In this test, hydrogen peroxide (H₂O₂) was added to simulate the situation where cells are subjected to external oxidative damage, and the resulting low mitochondrial activity was observed. Here, the lower the proportion of low mitochondrial activity calculated in this test, the better, indicating that Limosilactobacillus reuteri TCI979 can increase the metabolic rate of cells and reduce the aging phenomenon caused by the accumulation of waste.

### 5-1. Materials and Instruments:

Cell line: mouse myoblasts C2C12 (hereinafter referred to as C2C12 cells for short) adopted. The C2C12 cells were purchased from the C2C12 cell line (Cat. 60083) of the Bioresource Collection and Research Center (BCRC).

Medium: Dulbecco's modified Eagle's medium adopted, purchased from Gibco, the United States, model Gat. 11965-092; 10% fetal bovine serum added, purchased from Gibco, the United States, model Gat. 10437-028; and 1% antibiotic-antifungal agent added, purchased from Gibco, the United States, model Gat.15240-062.

Reagents: PBS buffer (purchased from Gibco, model Gat.14200-075), trypan blue dead cell stain (purchased from Lonza, model Cat. 17-942E), trypsin: 10× Trypsin-EDTA (purchased from Gibco), MitoScreen flow cytometer mitochondrial membrane potential detection kit (BD; Cat. BDB551302) including: JC-1 dye, and 10× Assay buffer; and hydrogen peroxide H₂O₂ (purchased from Sigma).

### 5-2. Test Procedure:

First, 1×10⁵ C2C12 cells were taken into a six-well cell culture plate containing 2 mL of medium per well and cultured at a constant temperature of 37° C. and 5% CO₂ for 24 hours. The C2C12 cells were divided into three groups: the experimental group, the blank group, and the control group.

Blank group: only the medium was added and cultured at a constant temperature of 37° C. and 5% CO₂ for 25 hours.

Control group: 1 mM of hydrogen peroxide was added and cultured at a constant temperature of 37° C. and 5% CO₂ for 25 hours.

Experimental group: the cell culture medium containing the TCI979 sample prepared in Example 2 with a concentration of 4% was added and cultured at a constant temperature of 37° C. and 5% CO₂ for 24 hours. Then 1 mM of hydrogen peroxide was added and the culture was continued at a constant temperature of 37° C. and 5% CO₂ for 1 hour.

The supernatant in the culture plate was removed and rinsed twice with 1 mL of 1× PBS solution. Then 200 μL of trypsin was added to each well and reacted in the dark for 5 minutes. After the reaction was completed, the cells in each well were collected into individual corresponding 1.5 mL centrifuge tubes, and the centrifuge tubes containing the cells were centrifuged at 400×g for 5 minutes. After centrifugation, the supernatant was removed, and then the cells were resuspended with 1 mL of PBS solution. Subsequently, the centrifuge tubes containing the cells were centrifuged again at 400×g for 5 minutes. After centrifuging again, the supernatant in each centrifuge tube was removed, and 100 μL of JC-1 working reagent was added to each centrifuge tube and allowed to stand for 15 minutes in the dark. After 15 minutes, each centrifuge tube was centrifuged at 400×g for 5 minutes. After centrifugation, the supernatant in each centrifuge tube was removed, and the cells were washed with 1 mL of buffer solution and then centrifuged at 400×g for 5 minutes. This step was repeated twice. After the second centrifugation, the supernatant in each centrifuge tube was removed, and the cells in each centrifuge tube were resuspended with 500 μL of 1× PBS solution (added with 2% FBS) to obtain the cell solution to be tested. Finally, the fluorescence signal (excitation light: 488 nm; scattered light: 527 nm & 590 nm) of the cell solution to be tested in each well was measured by a flow cytometer, and the mitochondrial membrane potential of the cells was calculated to analyze the mitochondrial activity.

### 5-3. Test Results:

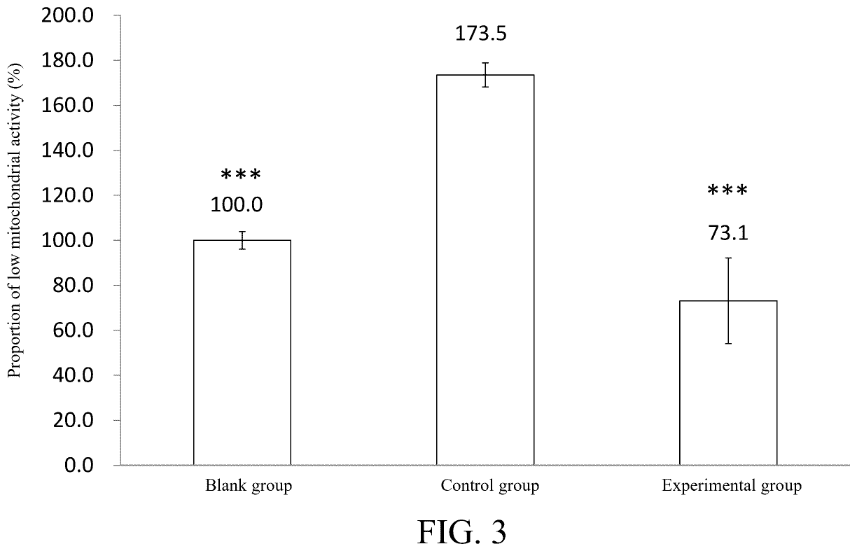

It should be particularly noted that the results shown in FIG. 3 were presented in relative multiples, i.e., the quantitative result of the blank group was regarded as 100, and the quantitative results of the experimental group and the control group were converted into the expression amount relative to that of the blank group. The Student T-Test was used to analyze whether there was a statistically significant difference. In the figure, “*” represents that the p-value is less than 0.05 relative to the control group, “**” represents that the p-value is less than 0.01 relative to the control group, and “***” represents that the p-value is less than 0.001 relative to the control group.

Please refer to FIG. 3. When the situation of low mitochondrial activity measured in the blank group was regarded as 100%, the situation of low mitochondrial activity in the control group relative to the blank group was 173.5%. It meant that the blank group showed the normal situation (without external oxidative damage), while in the control group, hydrogen peroxide (H₂O₂) was added to simulate the situation where cells are subjected to external oxidative damage, and the resulting low mitochondrial activity in the control group is significantly much more pronounced.

In contrast, the situation of low mitochondrial activity in the experimental group relative to the blank group was 73.1%, and it was significantly reduced by 100.4% compared with the control group. It indicates that Limosilactobacillus reuteri TC1979 can effectively assist cells in resisting the low mitochondrial activity caused by hydrogen peroxide and restore the performance of low mitochondrial activity caused by external oxidative damage.

### Example 6: Telomerase Activity Test

Telomerase is a reverse transcriptase with an RNA segment. It is generally believed that it can repair the DNA of telomeres through reverse transcription, extend the length of telomeres, and maintain the integrity and stability of chromosomes. In this test, the ability of cells to maintain telomere length is used to further infer the effect of telomerase on cell proliferation, aging, or potential stemness characteristics. The higher the telomerase activity, the stronger the ability of repairing the DNA at the ends of chromosomes.

### 6-1. Materials and Instruments:

Cells: human chorionic stromal cells (cbMSC-hTERT-RFP, BCRC, No. 60605, hereinafter referred to as stromal cells for short).

Medium: 80% minimum essential medium (purchased from Eagle), added with 20% Fetal Bovine Serum (FBS, purchased from Gibco), 1% penicillin-streptomycin (purchased from Gibco), and 1 mM sodium pyruvate (purchased from Thermo Fisher), and 4 ng/ml human Basic Fibroblast Growth Factor (bFGF, purchased from Gibco, 13256-029).

PBS buffer and DPBS buffer (purchased from Gibco).

Flow cytometer (purchased from BD Company, model Accuri™ C6 Plus).

### 6-2. Test Procedure:

First, 1×10⁵ stromal cells were seeded into a 24-well cell culture plate and cultured at 37° C. and 5% CO₂ for 24 hours. After the stromal cells in each well were cultured, the stromal cells were separated into a blank group and an experimental group.

Blank group: only the medium was added and cultured at 37° C. for three days.

Experimental group: the cell culture medium containing the TCI979 sample prepared in Example 2 with a concentration of 0.125% was added and cultured at 37° C. for three days.

Next, the supernatant of the cells in each group was removed, washed twice with 1× PBS buffer, trypsin was added to detach the stromal cells from the culture plate and transfer them to 1.5 mL centrifuge tubes. Then 2% fetal bovine serum was added to neutralize the reaction, and the tubes were centrifuged (400×g) for 5 minutes. After centrifugation, the supernatant was removed, washed again with 1× PBS buffer, and centrifuged again (400×g) for 5 minutes. Medium was added to each centrifuge tube to resuspend the stromal cells, forming the cell solution to be tested.

The flow cytometer (with a set wavelength of 632-647 nm) was used to detect the red fluorescence signals in the cell solution to be tested of each group. The fluorescence intensity can reflect the state of telomerase activity and was converted into relative values.

### 6-3. Test Results:

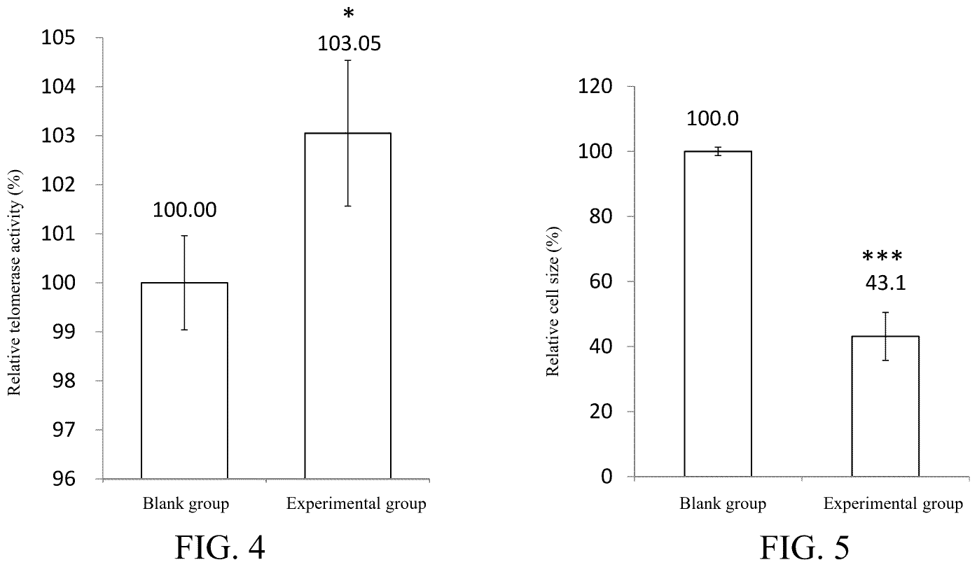

It should be particularly noted that the results shown in FIG. 4 were presented in relative multiples, i.e., the quantitative result of the blank group was regarded as 100, and the quantitative results of the experimental group and the control group were converted into the expression amount relative to that of the blank group. The Student T-Test was used to analyze whether there was a statistically significant difference. In the figure, “*” represents that the p-value is less than 0.05 relative to the blank group, “**” represents that the p-value is less than 0.01 relative to the blank group, and “***” represents that the p-value is less than 0.001 relative to the blank group.

Please refer to FIG. 4. As shown in FIG. 4, compared with 100% of the blank group, the relative telomerase activity of the experimental group was improved to 103.05%. It can be seen that Limosilactobacillus reuteri TC1979 can effectively improve the telomerase activity by 3.9% and delay the rate of telomere shortening.

### Example 7: Test on Cell Size and Morphology Under Natural Aging

In this test, the effect of the Limosilactobacillus reuteri on the natural aging of cells was observed. Changes in cell size and morphology are one of the indicators for observing cell aging in in vitro cell experiments. When cells swell and the number of cell filaments increases, it indicates severe cell aging. Generally, the main causes of natural cell aging are the continuous shortening of telomeres due to cell division and the increasing accumulation of reactive oxygen species within the cells.

### 7-1. Description of Test Materials and Equipment:

Cell line: human vascular endothelial cells (EA.hy926, hereinafter referred to as endothelial cells for short) adopted, purchased from ATCC, CRL-2922.

Medium: 90% DMEM medium (Dulbecco's Modified Eagle Medium, brand: Gibco), 10% Fetal Bovine Serum (brand: Gibco), and 1% antibiotics (penicillin-streptomycin, brand: Gibco).

Reagents: Dulbecco's phosphate buffered saline (brand: Gibco, hereinafter referred to as PBS for short), and trypsin.

Stains: ActinRed™ 555 ReadyProbes™ Reagent (Thermo; Cat. R37112), and Hoechst 33342 (Thermo; Cat. 62249).

Equipment: fluorescence microscope (brand: ZEISS, Vert.A1), and flow cytometer (purchased from BD Company, model Accuri™ C6 Plus).

### 7-2. Test Procedure:

First, the endothelial cells were seeded into each well of a 6-well culture plate containing 2 mL of medium at a cell count of 2×10⁵ cells per well and cultured at 37° C. for 24 hours. Then the cells were divided into a blank group and an experimental group.

Experimental group: cultured with the medium containing the TCI979 sample cultivated in Example 2 with a concentration of 0.125% at 37° C. for 7 days. The medium was changed every 2-3 days during the 7-day cell treatment period.

Blank group: only the medium was added and cultured at 37° C. for 7 days. The medium was also changed every 2-3 days during the 7-day cell treatment period.

First, trypsin was added to detach the endothelial cells from the culture plate. ⅘ of the cells were transferred to 1.5 mL centrifuge tubes. Each centrifuge tube was used to wash the endothelial cells with DPBS and then centrifuged at 400×g for 5 minutes. After centrifugation, the supernatant was removed, and 200 μL of DPBS was added to each centrifuge tube to resuspend the cells, forming the cell solution to be tested. The flow cytometer was used to measure each cell solution to be tested. First, cell size distribution was quantified. Then, software was used to select the larger cell populations in each group, and the percentage within the selected range was recorded. The results are shown in FIG. 5.

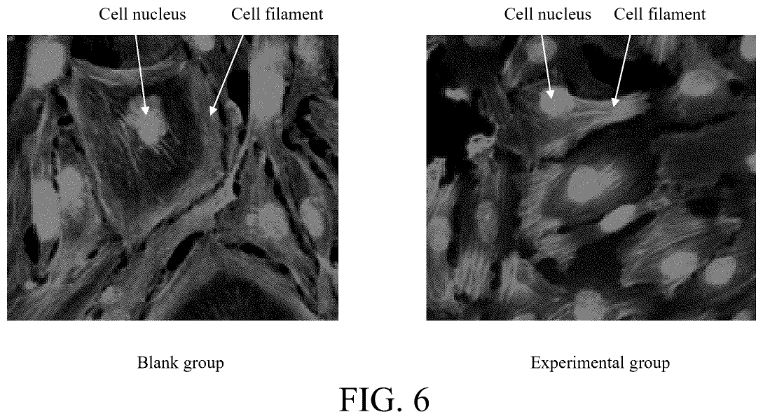

The remaining ⅕ of the cells were seeded into a 24-well culture plate. 1 drop of ActinRed™ 555 ReadyProbes™ Reagent and 300 μL of Hoechst 33342 (diluted 1:20000) were added to each well for staining, and the reaction was carried out in the dark for 15 minutes. Observation and photographing were performed with a fluorescence microscope in the dark. The results are shown in FIG. 6.

### 7-3. Test Results:

Here, the results shown in FIG. 5 were presented in relative multiples, i.e., the quantitative result of the blank group was regarded as 100, and the quantitative result of the experimental group was converted into the expression amount relative to that of the blank group. The Student T-Test was used to analyze whether there was a statistically significant difference. In the figure, “*” represents that the p-value is less than 0.05 relative to the blank group, “**” represents that the p-value is less than 0.01 relative to the blank group, and “***” represents that the p-value is less than 0.001 relative to the blank group.

Referring to FIG. 5, when the cell size of the blank group was regarded as 100%, the relative cell size of the experimental group was significantly reduced to 43.1%. It can be seen that Limosilactobacillus reuteri TC1979 can effectively inhibit cell swelling and has the function of preventing cell aging.

Referring to FIG. 6, by observing the blank group and the experimental group within the same selected range, it could be seen that the number of cell nuclei in the blank group was small, the overall cells were significantly swollen, and the number of cell filaments was large. In contrast, within the selected range of the experimental group, the number of cell nuclei was large, the cells were in an elongated state without swelling, and the number of cell filaments was small and the cell filaments were dense. It can be seen that the Limosilactobacillus reuteri can effectively inhibit cell swelling and the excessive proliferation of cell filaments and has the function of preventing cell aging.

### Example 8: Test on Cell Size and Morphology Under External Oxidative Damage

In this test, hydrogen peroxide (H₂O₂) was added to simulate the situation where cells are subjected to external oxidative damage, and the resulting changes in cell size and morphology were observed.

### 8-1. Description of Test Materials and Equipment:

Cell line: human vascular endothelial cells (EA.hy926, hereinafter referred to as endothelial cells for short) adopted, purchased from ATCC, CRL-2922.

Medium: 90% DMEM medium (Dulbecco's Modified Eagle Medium, brand: Gibco), 10% Fetal Bovine Serum (brand: Gibco), and 1% antibiotics (penicillin-streptomycin, brand: Gibco).

Reagents: Dulbecco's phosphate buffered saline (brand: Gibco, hereinafter referred to as PBS for short), trypsin, and hydrogen peroxide H₂O₂ (purchased from Sigma).

Stains: ActinRed™ 555 ReadyProbes™ Reagent (Thermo; Cat. R37112), and Hoechst 33342 (Thermo; Cat. 62249).

Equipment: fluorescence microscope (brand: ZEISS, Vert.A1), and flow cytometer (purchased from BD Company, model Accuri™ C6 Plus).

### 8-2. Test Procedure:

First, the endothelial cells were seeded into each well of a 6-well culture plate containing 2 mL of medium at a cell count of 2×10⁵ cells per well and cultured at 37° C. for 24 hours. Then the cells were divided into a blank group, a control group, and an experimental group.

Blank group: only the medium was added and treated at 37° C. for 3 hours.

Control group: only the medium was added and treated at 37° C. for 1 hour, and then a cell culture medium containing 30 μM of hydrogen peroxide was added and treated at 37° C. for 2 hours.

Experimental group: the cell culture medium containing the TCI979 sample prepared in Example 2 with a concentration of 0.125% was added and treated at 37° C. for 1 hour, and then 30 μM of hydrogen peroxide was added and treated at 37° C. for 2 hours.

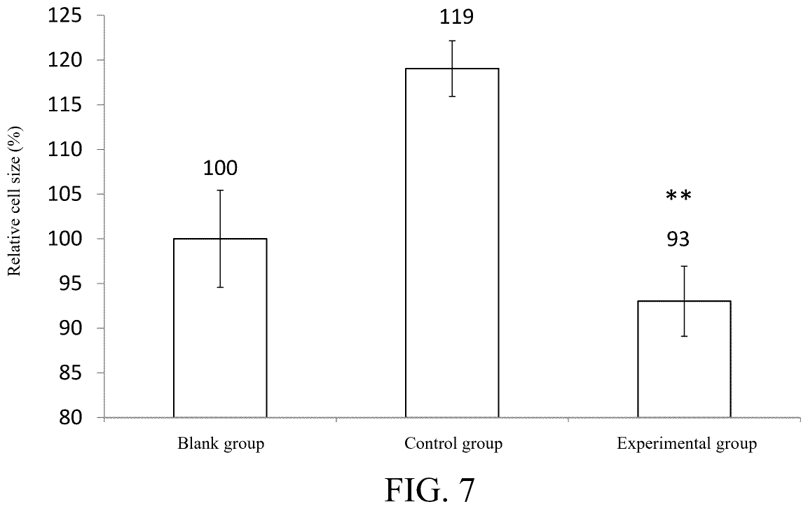

Next, trypsin was added to detach the endothelial cells from the culture plate. The cells were transferred to 1.5 mL centrifuge tubes. Each centrifuge tube was used to wash the endothelial cells with DPBS and then centrifuged at 400×g for 5 minutes. After centrifugation, the supernatant was removed, and 200 μL of DPBS was added to each centrifuge tube to resuspend the cells, forming the cell solution to be tested. The flow cytometer was used to measure each cell solution to be tested. First, cell size distribution was quantified. Then, software was used to select the larger cell populations in each group, and the percentage within the selected range was recorded. The results are shown in FIG. 7.

### 8-3. Test Results:

It should be particularly noted that the results shown in FIG. 7 were presented in relative multiples, i.e., the quantitative result of the blank group was regarded as 100, and the quantitative results of the experimental group and the control group were converted into the expression amount relative to that of the blank group. The Student T-Test was used to analyze whether there was a statistically significant difference. In the figure, “#” represents that the p-value of the control group is less than 0.05 relative to the blank group, “##” represents that the p-value of the control group is less than 0.01 relative to the blank group, and “###” represents that the p-value of the control group is less than 0.001 relative to the blank group; “*” represents that the p-value of the experimental group is less than 0.05 relative to the control group, represents that the p-value of the experimental group is less than 0.01 relative to the control group, and “***” represents that the p-value of the experimental group is less than 0.001 relative to the control group.

Referring to FIG. 7, when the cell size of the blank group was regarded as 100%, the relative cell size of the cells in the control group under oxidative damage was significantly increased to 119%, while the relative cell size of the experimental group was significantly reduced to 93%. That is, Limosilactobacillus reuteri TCI979 can effectively resist the cell aging caused by external oxidative damage. It can be seen that Limosilactobacillus reuteri TCI979 can effectively inhibit cell swelling and has the function of preventing cell aging.

### Example 9: Human Test

9-1. Sample: capsules prepared from the Limosilactobacillus reuteri bacterial powder prepared in Example 2 adopted, each containing 100 mg of TC1979 bacterial powder.

9-2. Subjects: 5 subjects. All the subjects were elderly individuals aged over 60 years.

9-3. Test Items: blood malondialdehyde (MDA), total antioxidant capacity (TAC), sulfur-containing compounds (f-Thiols), upper limb muscle endurance, and lower limb muscle endurance.

The measurements of blood malondialdehyde (MDA), total antioxidant capacity (TAC), and sulfur-containing compounds (f-Thiols) were commissioned to Liren Medical Laboratory after blood sampling.

The test method for upper limb muscle endurance: the number of times each subject lifted a dumbbell with the dominant hand within 30 seconds, where lifting a dumbbell means bending and then straightening the elbow while holding the dumbbell in the palm. The dumbbells used were 5 pounds for women and 8 pounds for men.

The test method for lower limb muscle endurance: the number of times each subject stood up and sat down from a chair within 30 seconds.

### 9-4. Test Procedure:

The 5 subjects were instructed to take capsules prepared from the Limosilactobacillus reuteri bacterial powder in Example 2, with a daily intake of 100 mg, and the intake was continued for eight weeks. Measurements were separately performed before the start of intake (i.e., week 0, also known as a blank group), after four weeks of intake (i.e., week 4, also known as experimental group A), and after eight weeks of intake (i.e., week 8, also known as experimental group B).

The values of blood malondialdehyde, total antioxidant capacity, and sulfur-containing compounds were calculated based on the reports provided by the medical laboratory and presented as the average values of the five subjects.

The measurement results of the upper limb muscle endurance and lower limb muscle endurance were based on the data of week 0, and the data of week 4 and week 8 were presented in relative multiples. It meant that the quantitative results of week 0 were regarded as 100%, and the quantitative results of week 4 and week 8 were converted into relative percentages, so as to more clearly present the improvement status of each test item from the start to the end of the test.

### 9-5. Test Results:

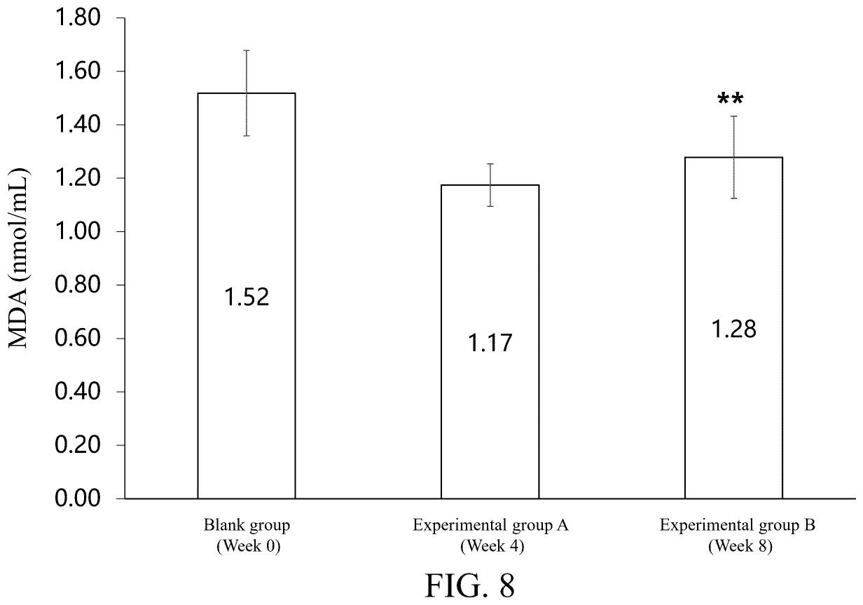

Please refer to FIG. 8. When the 5 subjects took 100 mg of the Limosilactobacillus reuteri daily while maintaining their regular diet and daily routine, the average malondialdehyde (MDA) concentration in blood decreased significantly from 1.52 mmol/L (blank group) to 1.17 mmol/L (experimental group A) after four weeks of intake. Although it slightly adjusted to 1.28 mmol/L (experimental group B) after eight weeks of intake, an overall downward trend could still be seen after taking Limosilactobacillus reuteri TCI979. The blood malondialdehyde (MDA) concentrations of all the 5 subjects decreased, i.e., the proportion of subjects with improvement reached 100%. It could be observed that after only eight weeks of using the Limosilactobacillus reuteri of the present invention, the difference in the average blood malondialdehyde before and after was 15.8%. It indicates that taking 100 mg of the Limosilactobacillus reuteri daily can effectively reduce the concentration of blood malondialdehyde in blood and improve the antioxidant capacity.

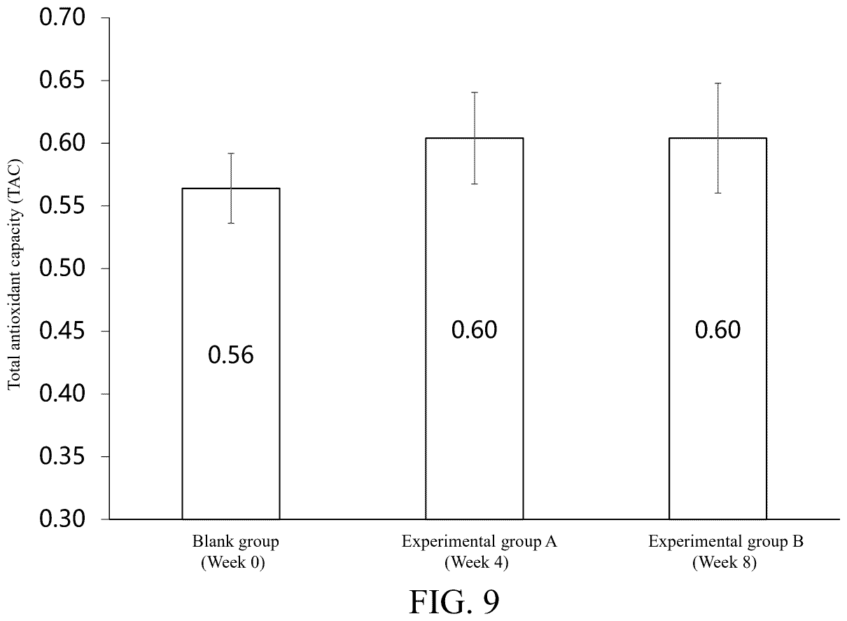

Please refer to FIG. 9. When the 5 subjects took 100 mg of the Limosilactobacillus reuteri daily while maintaining their regular diet and daily routine, the average total antioxidant capacity (TAC) increased from 0.56 (blank group) to 0.60 (experimental group A) after four weeks of intake, and remained stable at 0.60 (experimental group B) after eight weeks of intake. An overall upward trend could be seen after taking Limosilactobacillus reuteri TC1979. The total antioxidant capacity of 3 out of the 5 subjects increased, i.e., the proportion of subjects with improvement reached 60%. It could be observed that after only eight weeks of using the Limosilactobacillus reuteri of the present invention, the difference in the average blood total antioxidant capacity before and after was 7.1%. It indicates that taking 100 mg of the Limosilactobacillus reuteri daily can effectively improve the antioxidant capacity.

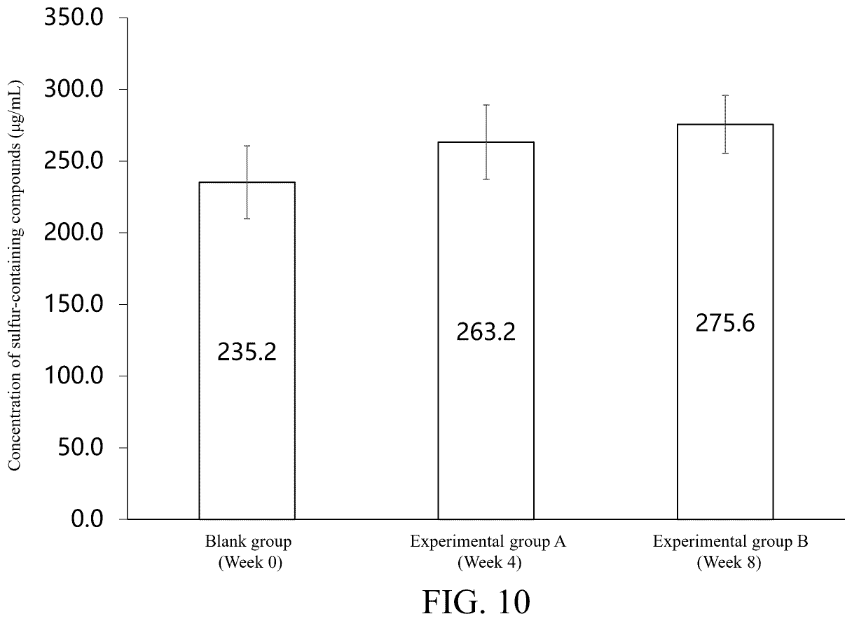

Please refer to FIG. 10. When the 5 subjects took 100 mg of the Limosilactobacillus reuteri daily while maintaining their regular diet and daily routine, the average concentration of sulfur-containing compounds (f-Thiols) in blood increased from 235.2 μg/mL (blank group) to 263.2 μg/mL (experimental group A) after four weeks of intake, and continued to increase steadily to 275.6 μg/mL (experimental group B) after eight weeks of intake. An overall upward trend could be seen after taking Limosilactobacillus reuteri TC1979. The concentrations of sulfur-containing compounds in blood of 4 out of the 5 subjects increased significantly, i.e., the proportion of subjects with improvement reached 80%. It could be observed that after only eight weeks of using the Limosilactobacillus reuteri of the present invention, the difference in the average concentration of sulfur-containing compounds in blood before and after was 17.2%. It indicates that taking 100 mg of the Limosilactobacillus reuteri daily can effectively increase the sulfur-containing compounds in blood and effectively mitigate the damage caused by reactive oxygen free radicals.

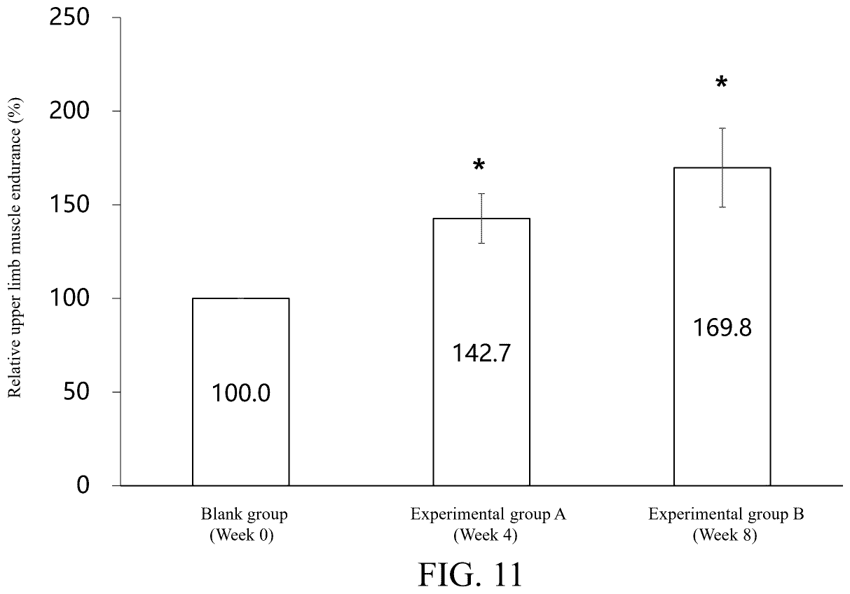

Please refer to FIG. 11. When the 5 subjects took 100 mg of the Limosilactobacillus reuteri daily while maintaining their regular diet and daily routine, the average upper limb muscle endurance increased from 100% (blank group) to 142.7% (experimental group A) after four weeks of intake, and continued to increase to 169.8% (experimental group B) after eight weeks of intake. A significant overall upward trend could be seen after taking Limosilactobacillus reuteri TC1979. The upper limb muscle endurance of all the 5 subjects increased significantly, i.e., the proportion of subjects with improvement reached 100%. It could be observed that after only eight weeks of using the Limosilactobacillus reuteri of the present invention, the difference in the average upper limb muscle endurance before and after was 69.8%. It indicates that taking 100 mg of the Limosilactobacillus reuteri daily can effectively improve the upper limb muscle endurance.

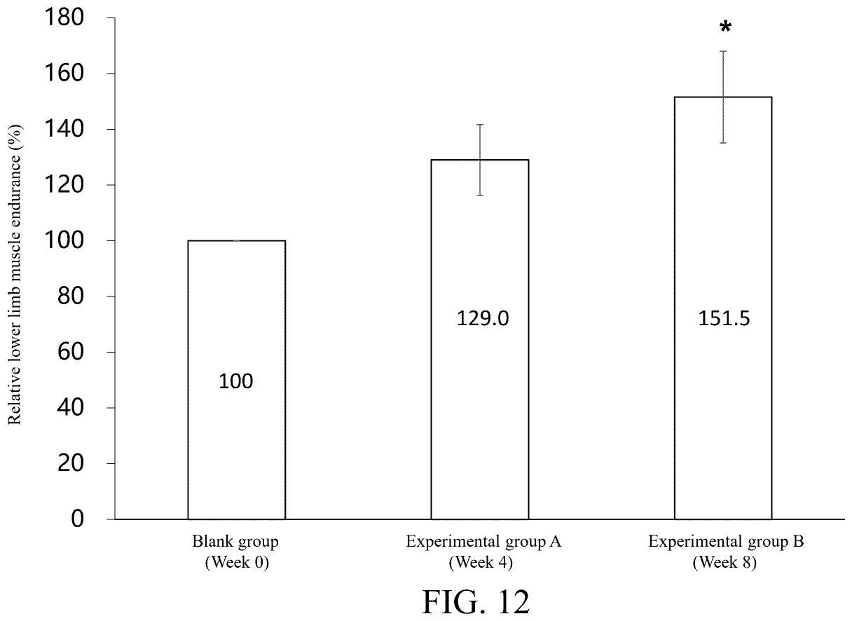

Please refer to FIG. 12. When the 5 subjects took 100 mg of the Limosilactobacillus reuteri daily while maintaining their regular diet and daily routine, the average lower limb muscle endurance increased from 100% (blank group) to 129.0% (experimental group A) after four weeks of intake, and continued to increase to 151.5% (experimental group B) after eight weeks of intake. A significant overall upward trend could be seen after taking Limosilactobacillus reuteri TC1979. The lower limb muscle endurance of 4 out of the 5 subjects increased significantly, i.e., the proportion of subjects with improvement reached 80%. It could be observed that after only eight weeks of using the Limosilactobacillus reuteri of the present invention, the difference in the average lower limb muscle endurance before and after was 51.5%. It indicates that taking 100 mg of the Limosilactobacillus reuteri daily can effectively improve the lower limb muscle endurance.

In summary, the Limosilactobacillus reuteri and/or the metabolite thereof in any of the embodiments may reduce the content of malondialdehyde in blood, improve the total antioxidant capacity or the content of sulfur-containing compounds in blood, inhibit the content of reactive oxygen, or reduce the apoptosis, thereby delaying the muscle loss. The Limosilactobacillus reuteri and/or the metabolite thereof in any of the embodiments may improve the mitochondrial activity of muscle cells, reduce the content of malondialdehyde in blood, improve the total antioxidant capacity or the content of sulfur-containing compounds in blood, or inhibit the content of reactive oxygen, thereby improving the muscle endurance. The Limosilactobacillus reuteri and/or the metabolite thereof in any of the embodiments may delay the rate of telomere shortening, improve the activity of telomerase, or inhibit the content of reactive oxygen, thereby delaying the cell aging.

Although the present invention has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. A method for delaying muscle loss, comprising administering to a subject in need thereof a composition containing an effective dose of a Limosilactobacillus reuteri and/or a metabolite thereof, wherein the Limosilactobacillus reuteri is Limosilactobacillus reuteri with the accession number BCRC911170 or DSM34539.

2. The method according to claim 1, wherein the Limosilactobacillus reuteri and/or the metabolite thereof are used to reduce a content of malondialdehyde in blood.

3. The method according to claim 1, wherein the Limosilactobacillus reuteri and/or the metabolite thereof are used to improve total antioxidant capacity (TAC) or a content of sulfur-containing compounds (f-Thiols) in blood.

4. The method according to claim 1, wherein the Limosilactobacillus reuteri and/or the metabolite thereof are used to inhibit a content of reactive oxygen.

5. The method according to claim 1, wherein the Limosilactobacillus reuteri and/or the metabolite thereof are used to reduce apoptosis.

6. A method for improving muscle endurance, comprising administering to a subject in need thereof a composition containing an effective dose of a Limosilactobacillus reuteri and/or a metabolite thereof, wherein the Limosilactobacillus reuteri is Limosilactobacillus reuteri with the accession number BCRC911170 or DSM34539.

7. The method according to claim 6, wherein the Limosilactobacillus reuteri and/or the metabolite thereof are used to improve mitochondrial activity of muscle cells.

8. The method according to claim 6, wherein the Limosilactobacillus reuteri and/or the metabolite thereof are used to reduce a content of malondialdehyde in blood.

9. The method according to claim 6, wherein the Limosilactobacillus reuteri and/or the metabolite thereof are used to improve total antioxidant capacity (TAC) or a content of sulfur-containing compounds (f-Thiols) in blood.

10. The method according to claim 6, wherein the Limosilactobacillus reuteri and/or the metabolite thereof are used to inhibit a content of reactive oxygen.

11. A method for delaying cell aging, comprising administering to a subject in need thereof a composition containing an effective dose of a Limosilactobacillus reuteri and/or a metabolite thereof, wherein the Limosilactobacillus reuteri is Limosilactobacillus reuteri with the accession number BCRC911170 or DSM34539.

12. The method according to claim 11, wherein the Limosilactobacillus reuteri and/or the metabolite thereof are used to delay a rate of telomere shortening.

13. The method according to claim 12, wherein the Limosilactobacillus reuteri and/or the metabolite thereof are used to improve activity of telomerase.

14. The method according to claim 11, wherein the Limosilactobacillus reuteri and/or the metabolite thereof are used to inhibit a content of reactive oxygen species.


SEQUENCE LISTING (1 sequence)

SEQ ID NO: 1 - DNA, 1050 bp, Limosilactobacillus reuteri
  source 1..1050: mol_type = genomic DNA
ccctgctgcgacattaagcggctccctccataaaggttaggccaccgactttgggcgttacaaactcccatggtgtgacgggcggtgtgtacaaggcccgggaacgtattcaccgcggcatgctgatccgcgattactagcgattccgacttcgtgtaggcgagttgcagcctacagtccgaactgagaacggctttaagagattagcttactctcgcgagtttgcgactcgttgtaccgtccattgtagcacgtgtgtagcccaggtcataaggggcatgatgatctgacgtcgtccccaccttcctccggtttgtcaccggcagtctcactagagtgcccaacttaatgctggcaactagtaacaagggttgcgctcgttgcgggacttaacccaacatctcacgacacgagctgacgacgaccatgcaccacctgtcattgcgtccccgaagggaacgccttatctctaaggttagcgcaagatgtcaagacctggtaaggttcttcgcgtagcttcgaattaaaccacatgctccaccgcttgtgcgggcccccgtcaattcctttgagtttcaaccttgcggtcgtactccccaggcggagtgcttaatgcgttagctccggcactgaagggcggaaaccctccaacacctagcactcatcgtttacggcatggactaccagggtatctaatcctgttcgctacccatgctttcgagcctcagcgtcagttgcagaccagacagccgccttcgccactggtgttcttccatatatctacgcattccaccgctacacatggagttccactgtcctcttctgcactcaagtcgcccggtttccgatgcacttcttcggttaagccgaaaggctttcacatcagacctaagcaaccgcctgcgctcgctttacgcccaataaatccggataacgcttgccacctacgtattaccgcggctgctggcacgtagttagccgtgactttctggttggataccgtcactgcgtgaacagttactctcacgcacgttcttctccacacagagc